(19) United States
(12) Patent Application Publication
(10) Pub. No.: US 2022/0203046 A1
(43) Pub. Date: Jun. 30, 2022

(54) AUTO PRIMING VENT PLUG

(71) Applicant: CareFusion 303, Inc., San Diego, CA (US)

(72) Inventors: Karthik BHASKAR, Coimbatore (IN); Manicavasagam GANAPATHY, Hosur (IN)

(21) Appl. No.: 17/139,555

(22) Filed: Dec. 31, 2020

(51) Int. Cl. A61M 5/36; A61M 5/14

(52) U.S. Cl. CPC A61M 5/36; A61M 2205/02; A61M 2005/1402; A61M 5/14

(57) ABSTRACT

An air venting device may include an adapter comprising a tubular body having a hollow interior defining a fluid flow path, and a vent plug detachably coupled to at least a portion of the tubular body. The vent plug may include an inner circumferential surface defining an internal chamber of the vent plug, and the fluid flow path of the adapter may fluidly communicate the adapter with the internal chamber of the vent plug. The vent plug may further include a superabsorbent polymer material disposed in the internal chamber. The superabsorbent polymer material may be configured to (i) absorb a liquid entering the internal chamber from the fluid flow path, and (ii) expand in volume as the liquid is absorbed into the superabsorbent polymer material. Air entrained in the liquid entering the internal chamber may be vented to an exterior of the air venting device via the vent plug.

### TECHNICAL FIELD

The present disclosure relates generally to systems and methods for venting air or gas out of a fluid tubing, and, in particular, to a vent plug that can be included within an extravascular system or an intravenous (“IV”) delivery system set to facilitate venting of air from the extravascular system or intravenous delivery system.

### BACKGROUND

Infusion therapy is one of the most common health care procedures. Hospitalized, home care, and other patients receive fluids, pharmaceuticals, and blood products via a vascular access device inserted into the vascular system. Infusion therapy may be used to treat an infection, provide anesthesia or analgesia, provide nutritional support, treat cancerous growths, maintain blood pressure and heart rhythm, or many other clinically significant uses.

Infusion therapy is facilitated by a vascular access device. The vascular access device may access a patient's peripheral or central vasculature. The vascular access device may be indwelling for short term (days), moderate term (weeks), or long term (months to years). The vascular access device may be used for continuous infusion therapy or for intermittent therapy.

A common vascular access device is a catheter that is inserted into a patient's vein. The catheter length may vary from a few centimeters for peripheral access to many centimeters for central access. The catheter may be inserted transcutaneously or may be surgically implanted beneath the patient's skin. The catheter, or any other vascular access device attached thereto, may have a single lumen or multiple lumens for infusion of many fluids simultaneously. A group of vascular access and other devices used to access the vasculature of a patient may be collectively referred to as an extravascular system.

One example of an extravascular system including a catheter is the BD NEXIVA™ Closed IV (intravenous) Catheter System, by Becton, Dickinson and Company. This system includes an over-the-needle, peripheral intravascular catheter made from polyurethane, another catheter used as an integrated extension tubing with a Y adapter and slide clamp, a vent plug, a Luer access port, and a passive needle-shielding mechanism.

The design of the BD NEXIVA™ IV catheter can be described as a closed system since it protects clinicians or operators from blood exposure during the catheter insertion procedure. Since the needle is withdrawn through a septum that seals, after the needle has been removed and both ports of the Y adapter are closed, blood is contained within the NEXIVA™ device during catheter insertion. The pressure exerted on the needle as it passes through the septum wipes blood from the needle, further reducing potential blood exposure. The slide clamp on the integrated extension tubing is provided to eliminate blood exposure when the vent plug is replaced with another vascular access device such as an infusion set connection or a Luer access port.

A current procedure of initiating the use of an extravascular system such as the BD NEXIVA™ Closed IV Catheter System is as follows. A device operator will insert the needle into the vasculature of a patient and wait for flashback of blood to travel into the device to confirm that the needle is properly located within the vasculature of the patient. The blood travels into and along the catheter of the device because a vent plug permits air to escape the device as blood enters the device. After an operator confirms proper placement, the operator clamps the catheter to halt the progression of blood through the catheter, removes the vent plug, replaces the vent plug with another vascular access device such as an infusion set connection or a Luer access port, unclamps the catheter, flushes the blood from the catheter back into the vasculature of the patient, and re-clamps the catheter.

### SUMMARY

Many current procedures like the procedure described above present challenges that need to be overcome. For example, the procedure may include an unnecessary number of steps and amount of time to simply insert and prepare an extravascular system for use within the vasculature of a patient. Further, by removing the vent plug, the fluid path of the system is temporarily exposed to potential contamination from the external environment of the extravascular system.

Rather than using a vent plug, some operators attempt to solve the problem above by simply loosening a Luer access device and permitting air to escape from the system during flashback and then tightening the Luer access device to stop blood from advancing along the catheter. Unfortunately, this procedure is also prone to user error, a lack of consistent and accurate control of blood flow through the system potentially leading to blood exposure and loss of body fluids, and unnecessary risk of contamination.

Thus, what are needed are improvements to many of the systems and methods described above. Such systems and methods can be improved by providing more efficient extravascular venting systems and methods.

In accordance with various embodiments of the present disclosure, an air venting device for facilitating self-priming of a fluid line may include an adapter for connecting vascular access devices and a vent plug. The adapter may include a tubular body having a hollow interior defining a fluid flow path, and the vent plug may be detachably coupled to at least a portion of the tubular body. The vent plug may include an inner circumferential surface defining an internal chamber of the vent plug, and the fluid flow path of the adapter may fluidly communicate the adapter with the internal chamber of the vent plug. The vent plug may further include a superabsorbent polymer material disposed in the internal chamber of the vent plug. The superabsorbent polymer material may be configured to (i) absorb a liquid entering the internal chamber from the fluid flow path, and (ii) expand in volume as the liquid is absorbed into the superabsorbent polymer material. Air entrained in the liquid entering the internal chamber may be vented to an exterior of the air venting device via the vent plug.

In accordance with various embodiments of the present disclosure, a method of assembling an auto-priming vent plug may include providing a substantially tubular body having an upper chamber, a lower chamber, a seating surface extending longitudinally in the lower chamber, and an inner circumferential surface defining an internal chamber of the tubular body. The method may further include coupling a perforated screen to the inner circumferential surface at a proximal end of the tubular body, and interposing a superabsorbent polymer material in the upper chamber between the perforated screen and the seating surface.

In accordance with various embodiments of the present disclosure, a vent plug may include a substantially tubular body having an upper chamber, a lower chamber and an inner circumferential surface defining an internal chamber of the tubular body, and a perforated screen coupled to the inner circumferential surface at a proximal end of the tubular body. The vent plug may further include a superabsorbent polymer material disposed in the upper chamber between the perforated screen and a proximal end of the lower chamber.

It is to be understood that both the foregoing general description and the following detailed description are exemplary and explanatory and are intended to provide further explanation of the subject technology as claimed. It is also to be understood that other aspects may be utilized, and changes may be made without departing from the scope of the subject technology.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a perspective view of an extravascular system having an adapter with a removable vent plug, in accordance with some embodiments of the present disclosure.

FIG. 2 is a cross-sectional view of an adapter, ventable end cap, and removable vent plug.

FIG. 3 is a cross-sectional view of an adapter and removable vent plug, in accordance with some embodiments of the present disclosure.

FIG. 4 is a cross-sectional view of an adapter and removable vent plug of FIG. 3, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Various embodiments of the present disclosure are generally directed to systems and methods for venting air or gas out of a fluid tubing, and, in particular, to a vent plug that can be included within an extravascular system or an intravenous delivery system set to facilitate venting of air from the extravascular system or intravenous delivery system.

According to various embodiments of the present disclosure, a luer integrated air venting system with a self-venting mechanism that permits escape of air during use which, typically, also prevents an outflow of fluid, such as blood is presented. As used herein, the term “venting mechanism” indicates one or more features or elements that provide venting of air, but which, typically, prevent liquid from passing through. The term “proximal” is used to denote a portion of a device, which during normal use, is nearest the user or clinician and furthest from the patient. The term “distal” is used to denote a portion of a device which, during normal use, is farthest from the user wielding the device and closest to the patient.

This invention may be suitable for use in any closed system luer connection application in which venting can facilitate self-priming, one example of a suitable application is an extravascular system, such as a Closed Intravenous (IV) Catheter System.

It should be noted that the vent media could be, for example, a distinct physical element such as a plug or insert, an integral portion of a device that has been treated such as by laser drilling or has been formed in whole or in part from a porous material, or a coating, layer, etc. formed by disposing a material onto the device, e.g., by dipping, coating, spraying or the like.

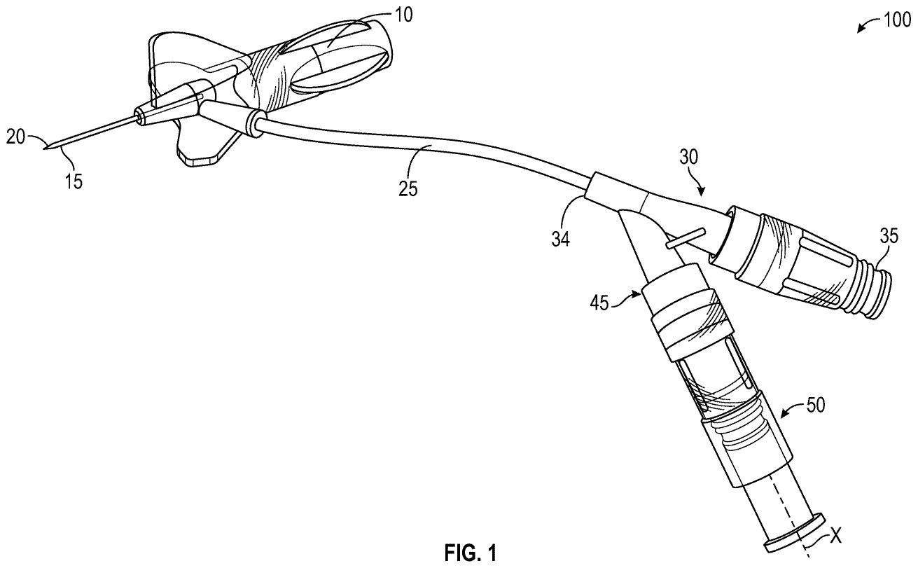

FIG. 1 is a perspective view of an extravascular system 100 having an adapter 30 with a removable vent plug 50, in accordance with some embodiments of the present disclosure. The extravascular system 100, may be a closed intravenous (IV) catheter system which is used to communicate fluid with the vascular system of a patient. As depicted, the extravascular system may include an intravascular needle 20, an over-the-needle, peripheral intravascular catheter 15, an integrated extension tubing 25 (also referred to herein as a catheter) with a Y-adapter 30. The extravascular system 100 may further have an air venting device 45 including a vent plug 50, a Luer access port 35, and a passive needle-shielding mechanism 10. Although a Y-adapter 30 is depicted, the various embodiments of the present disclosure are not limited to this configuration. In some embodiments, any adapter used to connect two or more vascular access devices may be used in place of the Y-adapter 30.

The extravascular system 100 may be referred to as a closed system since it protects clinicians or operators from blood exposure during the catheter 15 insertion procedure. Since the needle 20 is withdrawn through a septum that seals after the needle 20 has been removed and both ports of the Y adapter 30 are closed, blood is contained within the system 100 during insertion of the catheter 15. The pressure exerted on the needle 20 as it passes through the septum wipes blood from the needle 20, further reducing potential blood exposure. A slide clamp (not shown) may be provided on the integrated extension tubing 25 to eliminate blood exposure when the vent plug 50 is replaced with another vascular access device such as an infusion set connection or another luer access port.

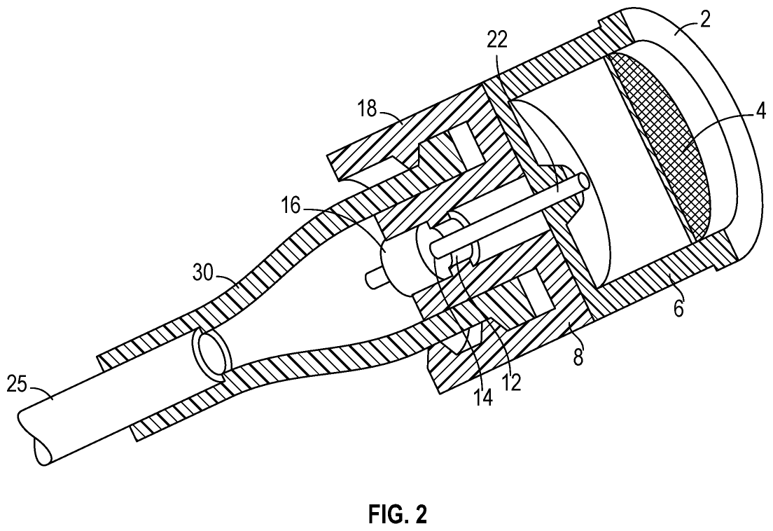

FIG. 2 is a cross-sectional view of an adapter 30, ventable end cap 18, and removable vent plug 2. As depicted, the ventable end cap 18 includes a body 8 having an open channel 12 containing an elastomeric septum 16. The septum 16 has a very small access hole 14 that is sealed under compression in the end cap 18 assembly. The septum access hole 14 allows a hollow cannula 22 from the removable vent plug 2 to pass, thereby, providing communication between the atmospheric pressure and the venous pressure of a patient and allowing air to vent and blood to flashback and be visible up the extension tubing 25 or other catheters attached to the extravascular system. The vent plug 2 may include a body 6, the attached cannula 22, and an air permeable material 4 or other air filter. The air permeable material 4 allows airflow to pass but prevents liquid from passing. For example, the air permeable material 4 may be an acrylic hydrophobic membrane, which permits air to escape the extravascular system as blood enters the system. The vent plug 2 described with reference to FIG. 2 allows an integrated catheter system to vent while remaining closed, thereby allowing flashback visualization of blood without exposing a physician to the blood. The system can easily be added to an existing integrated catheter system with a luer adapter.

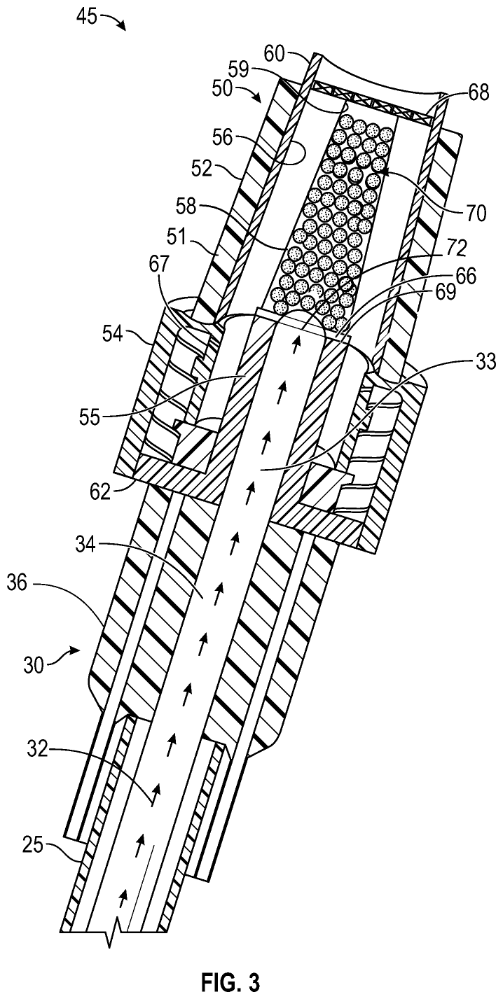

FIG. 3 is a cross-sectional view of an adapter 30 and vent plug 50 of an air venting device 45, in accordance with some embodiments of the present disclosure. As depicted, the adapter 30 may be in the form of a tubular body 36 having a hollow interior defining a fluid flow path 34 therein. The vent plug 50 may be detachably coupled to at least a portion of the tubular body 36. In some embodiments, the vent plug 50 may be in the form of a substantially tubular body 51 including an inner circumferential surface 59 defining an internal chamber 58 of the vent plug 50. Accordingly, the tubular body 36 defines the internal longitudinal passageway or bore 58 extending from a proximal end 60 to a distal end 62 and fluidly connected to the fluid flow path 34. The fluid flow path 34 of the adapter 30 may fluidly communicate the adapter 30 with the internal chamber 58 of the vent plug 50 so as to allow a fluid, e.g., blood from a patient to flow into the vent plug 50 from extension tubing 25 which may be coupled or otherwise connected to a peripheral intravascular catheter 15 (as illustrated in FIG. 1).

In accordance with various embodiments of the present disclosure, the vent plug 50 may have an upper chamber 52 and a lower chamber 54 disposed axially opposite and connected to the upper chamber 52. For example, the lower chamber 54 may be formed from at least one lower wall extending away from the upper chamber 52. As depicted, the vent plug 50 may be formed with a raised pedestal portion 55 projecting proximally and longitudinally from the distal end 62 of the vent plug 50 in the lower chamber 54. The raised pedestal portion 55 may include an inlet port 72 of the vent plug 50 and define a portion of the flow path 34 and terminate in a seating surface 69. For example, the seating portion 69 may define a fluid channel 33, which forms a part of the flow path 34 that is fluidly communicated with a lumen of a needle device (e.g., needle 20) configured to be inserted into a patient. Accordingly, the internal chamber 58 having the superabsorbent polymer material 70 may be fluidly connected with the flow path 34 via the port 72, and as such the superabsorbent polymer material 70 may come into contact with and be exposed to the patient's blood.

In some embodiments, the vent plug 50 may include a perforated screen 68 disposed at the proximal end 60 of the vent plug upper chamber 52. A superabsorbent polymer material 70 may be disposed in the internal chamber 58 of the vent plug 50 between the perforated screen 68 and the seating surface 69. As shall be described in further detail below, the superabsorbent polymer material 70 may be configured to (i) absorb a liquid (e.g., blood) entering the internal chamber 58 from the fluid flow path 34, and (ii) expand in volume upon contact with the liquid in the internal chamber. The perforated screen 68 may allow air or gas entrained in the liquid to vent out through the proximal end 60 of the vent plug 50. In the aforementioned configuration, the superabsorbent polymer material 70 absorbs and traps the liquid (e.g., blood) and expands to obstruct further blood flow into the vent plug 50 while allowing the entrained air or gas to vent out of the vent plug 50 via the perforated screen 68.

In some embodiments, the superabsorbent polymer material 70 may be a sponge, a sheet, or a mesh material. In other embodiments, the superabsorbent polymer material 70 may be a powder or granulated material. In these embodiments, the vent plug may further include a porous membrane 66 disposed on the seating surface 69 of the raised pedestal portion 54. The superabsorbent polymer material 70 may be interposed between the perforated screen 68 and the porous membrane 66 and the porous membrane 66 may serve as to prevent the powder or granulated superabsorbent polymer material from entering the fluid channel 33 and flow path 34 via the vent plug inlet port 72. In the embodiments where the superabsorbent polymer material 70 is not in a powder or granulated form, but is instead in the form of a sponge, a sheet, or a mesh material, the porous membrane 66 may be omitted. However, the various embodiments of the present disclosure are not limited to the aforementioned configuration. In some embodiments, the porous membrane 66 may be included where the superabsorbent polymer material 70 is in the form of a sponge, a sheet, or a mesh material.

In accordance with some embodiments of the present disclosure, the super absorbent polymer material may be impregnated on a base material. In these embodiments, the base material impregnated with the super absorbent polymer may replace the super absorbent polymer material 70 and/or the porous membrane 66. For example, the base material impregnated with the super absorbent polymer may be disposed on the seating surface 69 over the vent plug inlet port 72 so allow the base material impregnated with the super absorbent polymer to be exposed to the patient's blood flowing in the fluid channel 33 via the vent plug inlet port 72.

According to various embodiments of the present disclosure, the superabsorbent polymer material may be formed of at least one or a combination of acrylic acid sodium salt, a polyacrylamide copolymer, an ethylene maleic anhydride copolymer, cross-linked carboxymethylcellulose, polyvinyl alcohol copolymers, cross-linked polyethylene oxide, or a starch grafted copolymer of polyacrylonitrile. In some embodiments, the superabsorbent polymer material may include one or more bio-compatible super absorbent polymers.

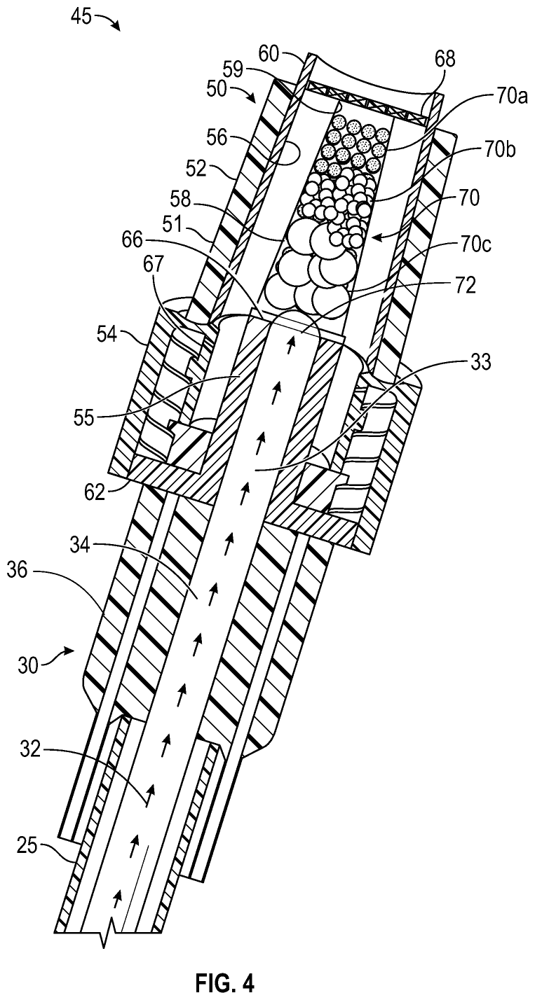

FIG. 4 is a cross-sectional view of an adapter and removable vent plug of FIG. 3, in accordance with some embodiments of the present disclosure. Referring to FIG. 4, with continued reference to FIG. 3, the super absorbent polymer material 70, upon exposure to the blood 32 flowing in the fluid channel 33, may immediately expand and swell in size from the configuration 70 illustrated in FIG. 3 to the configuration 70a, 70b, 70c illustrated in FIG. 4 as the super absorbent polymer material 70 absorbs the blood 32. As illustrated, the super absorbent polymer material 70 may swell from the seating surface 69 upwards to the proximal end 60 of the vent plug as the blood 32 is absorbed into and travels though the superabsorbent polymer material 70. For example, as depicted, the particles or structure of the superabsorbent polymer material 70 may swell from an initial non-liquid contact state 70a to a liquid absorption state 70c at the initial point of contact with the liquid at the seating surface 69. State 70b merely illustrates a transitionary state between the liquid absorption state 70c and the initial non-liquid contact state 70a.

In some embodiments, as described above, the perforated screen 68 and the porous membrane 66 may be separated from each other with the superabsorbent polymer material 70 interposed therebetween. The perforated screen 68 and the porous membrane 66 are formed in the internal chamber 58 of the vent plug through which air or other gas existing in the fluid 32 flowing in the fluid flow path 34 is desired to be vented from the system 100 to the external atmosphere via the vent plug 50. Referring to FIGS. 3 and 4, with continued reference to FIG. 1, in operation, a clinician/nurse, or other operator will insert the needle 20 into the vasculature of a patient and wait for flashback of blood to travel into the system 100 to confirm that the needle 20 is properly located within the vasculature of the patient. The blood 32 travels into and along the catheter 15 in the space between the needle 20 and the catheter 15. This occurs because the vent plug 50 permits air to escape the system 100 as blood enters the system 100. The vent plug of the various embodiments of the present disclosure utilizes the superabsorbent polymer material 70 along with the perforated screen 68 and an optional porous membrane 66 (depending on the form of the superabsorbent polymer material 70) to allow air to escape the system 100 as blood enters the system 100 as described below.

As blood flows from the vasculature of the patient into the system 100, air particles or bubbles may become trapped in the blood 32 stream as the blood travels into and along the catheter 15, into the extension tubing 25, and into the adapter 30. As the fluid 32 (e.g., the patient's blood 32) in the flow path 34 enters the vent plug 50 via the vent plug inlet port 72 and contacts the superabsorbent polymer material 70, the superabsorbent polymer material 70 absorbs the fluid 32, expands, and swells upon contact with the fluid 32. For example, in some embodiments, the superabsorbent polymer material 70 may expand at least 300% in volume upon contacting and absorbing the fluid. The aforementioned configuration is advantageous in that the superabsorbent polymer material—once expanded—acts as a stop or obstruction for further fluid (e.g., blood) to enter the vent plug 50 from the flow path 34 while at the same time allowing venting of the air or gas in the system 100 out of the vent plug 50 via the perforated screen 68.

In some embodiments, the porous membrane 66 may function as a stop for preventing the superabsorbent polymer material from entering the fluid flow path 34 of the adapter 30 via the vent plug inlet port. Accordingly, the porous membrane 66 may allow weeping of the fluid 32 under pressure from the fluid flow path 34 into the superabsorbent polymer material 70. The perforated screen 68 positioned at the proximal end 60 of the vent plug 50 may provide a second stop capable of preventing the superabsorbent polymer material 70 from expanding out of the vent plug 50 while allowing the air or other gas to be vented out of the system 100 through the perforated screen 68 at the proximal end 60 of the vent plug 50. After the operator, or other user, confirms proper placement, and after adequate venting of the system 100 has occurred, the clinician/nurse or other user may clamp the tubing 25 to halt the progression of blood through the catheter 15, remove the vent plug 50, unclamp the tubing 25, flush the blood from the catheter 15 back into the vasculature of the patient, and re-clamp the tubing 25.

According to various aspects of the present disclosure, a method of assembling an air vent plug 50 for facilitating priming of a fluid line may include providing a substantially tubular body 51 having an upper chamber 52, a lower chamber 54, a seating portion having a seating surface 69 extending longitudinally in the lower chamber 54, and an inner circumferential surface 59 defining an internal chamber 58 of the tubular body 51. The method may further include coupling a perforated screen 68 to the inner circumferential surface 59 at a proximal end 60 of the tubular body 51 and interposing the superabsorbent polymer material 70 in the upper chamber 52 between the perforated screen 68 and the seating surface 69. In some embodiments, the method may further include coupling a porous membrane 66 to the seating surface 69 in such a manner that the superabsorbent polymer material 70 is disposed between the porous membrane 66 and the perforated screen 68.

The vent plug 50 of the various embodiments of the present disclosure is advantageous over existing vent plugs as the vent plug 50 utilizes the superabsorbent polymer material 70 along with a perforated screen 68 and optional porous membrane 66 (depending on the form of the superabsorbent polymer material 70) to vent air out of the system 100 as described above. In particular, the aforementioned configuration of the vent plug 50 is advantageous in the superabsorbent polymer material 70 is capable of swelling, expanding, or otherwise inflating by at least 300% shortly after coming into contact with the fluid 32 (i.e., the blood 32 and air or gas particles) in the flow path 34. Upon contact with the fluid, the superabsorbent polymer material 70 may absorb the fluid and swell in volume, thereby obstructing further fluid flow into the vent plug 50. In some embodiments, the superabsorbent polymer material is configured to hold the fluid (e.g., blood of the patient) for a minimum of 15 seconds while allowing any air or gas in the system 100 to vent outside the system 100 via the perforated screen 68 of the vent plug 50. In some embodiments, the superabsorbent polymer material is capable of holding the fluid (e.g., blood of a patient) for a much longer period in presence of the fluid than the currently existing vent plugs which utilize a hydrophobic membrane.

In contrast, as described above, currently existing vent plugs, e.g., the vent plug 2 illustrated in FIG. 2 utilizes a hydrophobic membrane 4, which is air or gas permeable material to allow airflow to pass but prevent liquid from passing through. This hydrophobic membrane 4 is commonly made in the form of an acrylic hydrophobic membrane which can be costly to obtain and manufacture, thereby adding to the overall cost of the vent plug 2. Accordingly, since the vent plug 50 utilizes the superabsorbent polymer material 70 instead of the costly acrylic hydrophobic membrane 4 for venting of air in the system 100, a cost to produce and manufacture the vent plug 50 is decreased compared to that of the currently existing vent plug 2 having the acrylic hydrophobic membrane 4.

Although the various embodiments of the vent plug 50 have been described with respect to an extravascular system (e.g., a closed IV catheter system) the various embodiments of the present disclosure are not limited to the aforementioned configuration. In some embodiments, the vent plug 50 may be included within an intravenous delivery system set to facilitate venting of air from the intravenous delivery system. An intravenous delivery system according to the invention is used broadly herein to describe components used to deliver the fluid to the patient, for use in arterial, intravenous, intravascular, peritoneal, and/or non-vascular administration of fluid. Of course, one of skill in the art may use an intravenous delivery system to administer fluids to other locations within a patient's body.

For example, in some embodiments, an intravenous delivery system may include a liquid source such as a liquid bag, a drip chamber used to determine the flow rate of fluid from the liquid bag, tubing for providing a connection between the liquid bag and the patient, and an intravenous access unit, such as a catheter that may be positioned intravenously in a patient. The intravenous delivery system may also include the Y-connector 30 that allows for the piggybacking of intravenous delivery systems and for the administration of medicine from a syringe into the tubing of the intravenous delivery system.

It is a generally good practice to remove air from intravenous delivery systems that access a patient's blood flow. While this concern is critical when accessing arterial blood, it is also a concern when accessing the venous side. Specifically, if air bubbles are allowed to enter a patient's blood stream while receiving the intravenous administration of fluids, the air bubbles can form an air embolism and cause serious injury to a patient.

Embodiments of the present invention may also be generally directed to an intravenous delivery system having the vent plug 50 that provides enhanced air venting. For example, the intravenous delivery system may have a liquid source containing a liquid to be delivered to a patient, tubing, and the vent plug 50. The tubing may have a first end connectable to the liquid source, and a second end connectable to the vent plug 50. In some embodiments, the distal end 62 of the vent plug 50 may be connectable to the proximal end of the IV tubing to receive the liquid from the liquid source. In some embodiments, the vent plug 50 may have a volume selected to enable the internal chamber 58 to receive a quantity of liquid from the IV tubing in which air or gas, if entrained in the liquid, is likely to reside after the tubing has been primed sufficiently to advance the liquid through the proximal end of the IV tubing.

During priming, the vent plug 50 including the perforated screen 68 and the superabsorbent polymer material 70 disposed in the internal chamber 58 between the perforated screen 68 and the seating surface 69 may be configured to (i) absorb the IV liquid entering the internal chamber 58 from the proximal end of the IV tubing, and (ii) expand in volume as the IV liquid is absorbed into the superabsorbent polymer material. The perforated screen 68 may allow the entrained air or gas to completely vent out through the proximal end 60 of the vent plug 50 until the IV liquid comes in contact with superabsorbent polymer. In the aforementioned configuration, the superabsorbent polymer material 70 absorbs and traps the liquid molecules and expands to obstruct further IV fluid flow into the vent plug 50 while allowing the entrained air or gas to vent out of the vent plug 50 via the perforated screen.

As used herein, the terms “medical connector,” “connector,” “fitting,” and any variation thereof refer to any device used to provide a fluid flow path between fluid lines coupled thereto. For example, the medical connector may be or include a bond pocket or other types of connectors. Additionally, the terms “medical connector,” “connector,” “fitting,” and any variation thereof refer to any device used to deliver liquids, solvents, or fluids to or from a patient under medical care. For example, the medical connector may be used for intravenous (IV) delivery of fluids, fluid drainage, oxygen delivery, a combination thereof, and the like to the patient.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An air venting device for facilitating self-priming of a fluid line, comprising:
an adapter for connecting vascular access devices, the adapter comprising a tubular body having a hollow interior defining a fluid flow path; and
a vent plug detachably coupled to at least a portion of the tubular body, the vent plug comprising:
an inner circumferential surface defining an internal chamber of the vent plug, wherein the fluid flow path of the adapter fluidly communicates the adapter with the internal chamber of the vent plug; and
a superabsorbent polymer material disposed in the internal chamber of the vent plug, the superabsorbent polymer material configured to (i) absorb a liquid entering the internal chamber from the fluid flow path, and (ii) expand in volume as the liquid is absorbed into the superabsorbent polymer material,
wherein air entrained in the liquid entering the internal chamber from the fluid flow path is vented to an exterior of the air venting device via the vent plug.

2. The air venting device of claim 1, further comprising a catheter having a needle to be inserted into a patient, the needle being fluidly communicated with the fluid flow path of the adapter and the internal chamber of the vent plug to transport the liquid to the vent plug, wherein the liquid absorbed by the superabsorbent polymer material comprises blood of the patient.

3. The air venting device of claim 1, wherein the vent plug further comprises a lower chamber, an upper chamber and a perforated screen disposed at a proximal end of the vent plug upper chamber, wherein the air is vented to the exterior of the venting device via the perforated screen.

4. The air venting device of claim 3, wherein the vent plug further comprises a porous membrane interposed between the upper and lower chambers, the porous membrane configured to permit the liquid with entrained air to flow into the internal chamber from the fluid flow path and to restrain the superabsorbent polymer material from entering the fluid flow path.

5. The air venting device of claim 4, wherein the superabsorbent polymer material is disposed between the porous membrane and the perforated screen.

6. The air venting device of claim 5, wherein the superabsorbent polymer material comprises at least one material selected from the group consisting of a sponge, a powder, a sheet, or a mesh.

7. The air venting device of claim 5, wherein the superabsorbent polymer material is impregnated on a base material.

8. The air venting device of claim 1, wherein the superabsorbent polymer material expands at least 300 percent in volume upon absorbing the liquid entering the internal chamber from the fluid flow path.

9. The air venting device of claim 1, wherein the superabsorbent polymer material comprises at least one material selected from the group consisting of acrylic acid sodium salt, a polyacrylamide copolymer, an ethylene maleic anhydride copolymer, cross-linked carboxymethylcellulose, polyvinyl alcohol copolymers, cross-linked polyethylene oxide, or a starch grafted copolymer of polyacrylonitrile.

10. A method of assembling an auto-priming vent plug, the method comprising the steps of:
providing a substantially tubular body having an upper chamber, a lower chamber, a seating surface extending longitudinally in the lower chamber, and an inner circumferential surface defining an internal chamber of the tubular body;
coupling a perforated screen to the inner circumferential surface at a proximal end of the tubular body; and
interposing a superabsorbent polymer material in the upper chamber between the perforated screen and the seating surface.

11. The method of claim 10, wherein the superabsorbent polymer material comprises at least one material selected from the group consisting of a sponge, a sheet, or a mesh material.

12. The method of claim 10, further comprising coupling a porous membrane to the seating surface, the superabsorbent polymer material disposed between the porous membrane and the perforated screen.

13. The method of claim 12, wherein the superabsorbent polymer material comprises a powder or granulated material.

14. The method of claim 10, wherein the superabsorbent polymer material comprises at least one material selected from the group consisting of acrylic acid sodium salt, a polyacrylamide copolymer, an ethylene maleic anhydride copolymer, cross-linked carboxymethylcellulose, polyvinyl alcohol copolymers, cross-linked polyethylene oxide, or a starch grafted copolymer of polyacrylonitrile.

15. A vent plug, comprising:
a substantially tubular body having an upper chamber, a lower chamber and an inner circumferential surface defining an internal chamber of the tubular body;
a perforated screen coupled to the inner circumferential surface at a proximal end of the tubular body; and
a superabsorbent polymer material disposed in the upper chamber between the perforated screen and a proximal end of the lower chamber.

16. The vent plug of claim 15, further comprising a seating surface extending axially in the lower chamber, the seating surface comprising a fluid channel fluidly communicated with a lumen of a needle device configured to be inserted into a patient.

17. The vent plug of claim 16, further comprising a porous membrane disposed on the seating surface, wherein the superabsorbent polymer material is interposed between the perforated screen and the porous membrane.

18. The vent plug of claim 17, wherein the superabsorbent polymer material comprises a powder or granulated material.

19. The vent plug of claim 15, wherein the superabsorbent polymer material comprises at least one material selected from the group consisting of a sponge, a sheet, or a mesh material.

20. The vent plug of claim 15, wherein the superabsorbent polymer material is impregnated on a base material.
